(19) United States
(12) Patent Application Publication
(10) Pub. No.: US 2008/0009538 A1
(43) Pub. Date: Jan. 10, 2008

(54) Methods and compositions for the treatment of urinary incontinence

(76) Inventor: Phil Skolnick, Edgewater, NJ (US)

Correspondence Address: BLACK LOWE & GRAHAM PLLC, Suite 4800, 701 Fifth Avenue, Seattle, WA, 98104, US

(21) Appl. No.: 11/384,219

(22) Filed: Mar. 17, 2006

Related U.S. Application Data
(60) Provisional application No. 60/664,002, filed on Mar. 21, 2005.

(51) Int. Cl. A61K 31/40; A61P 13/10

(52) U.S. Cl. 514/412

(57) ABSTRACT

Methods and compositions containing bicifadine are provided for the prevention and treatment of lower urinary tract disorders in mammalian subjects. The methods and compositions may be used to prevent or treat urinary incontinence, urinary urgency, nocturia, and enuresis associated with neurogenic and non-neurogenic overactive bladder, interstitial cystitis, prostatitis, prostadynia, and benign prostatic hyperplasia, among other conditions. Additional compositions and methods are provided which employ bicifadine in combination with a second anti-incontinence agent, or a different therapeutic agent to yield more effective anti-incontinence treatment tools, and/or dual activity therapeutic methods and formulations useful to prevent or reduce urinary incontinence and one or more additional symptoms such as urinary urgency, overflow, frequency, or pain in mammalian subjects.

### REFERENCE TO RELATED APPLICATIONS

This application is related to and claims priority from U.S. Provisional Application 60/664,002, filed Mar. 21, 2005, the disclosure of which Provisional Application is incorporated herein by reference in its entirety.

### TECHNICAL FIELD

The present invention relates to methods and compositions for treating disorders of the lower urinary tract in mammalian subjects. More specifically, the invention relates to methods and compositions for treating and/or preventing urinary incontinence and related conditions in mammals.

### BACKGROUND

In mammals, the lower urinary tract stores and periodically eliminates urine produced by the kidneys. The ability to store and release urine depends on the activity of both smooth and striated muscles in the urinary bladder, urethra and urethral sphincter. These structures form a functional unit controlled by a complex interplay between the central and peripheral nervous systems, as well as local regulatory factors.

The normal urination, or micturition, reflex is a two-phase cycle mediated by a spinobulbospinal pathway through relay centers in the brain. Bladder emptying and filling are regulated by afferent signaling in parasympathetic, sympathetic and somatic nerves. These nerves maintain the bladder in a relaxed state enabling filling and storage, or initiate micturition by relaxing the outflow region and contracting the bladder smooth muscle. More specifically, parasympathetic neurons mediate contraction of the detrusor smooth muscle to relax the outflow region. Postganglionic neurons in the pelvic nerve mediate excitatory input to the detrusor smooth muscle by releasing acetylcholine, which acts on muscarinic receptors at the neuromuscular junction. Sympathetic neurons inhibit the parasympathetic pathways at the spinal and ganglion levels, mediating contraction of the bladder base and the urethra. Sensory neurons, including myelinated Aδ-fibers and unmyelinated C-fibers, convey information from receptors in the bladder regarding filling and chemical irritation. Additionally, alpha receptors located in the neck of the bladder are stimulated during the filling phase to contract and keep the bladder neck closed, and are inhibited during the emptying phase to relax and open the bladder and urethra. Beta receptors located in the bladder are stimulated during the filling phase to relax muscles and during the emptying phase to contract the bladder. Cholinergic receptors located throughout the bladder are inhibited during the filling phase to relax muscles, and are stimulated during the emptying phase to strengthen contraction of the bladder.

Normally, the bladder is able to hold and pass 300-400 ml or urine at a time, and is usually emptied 4-5 times during the day and no more than once at night. This storage and voiding pattern can be profoundly disrupted in individuals who suffer from lower urinary tract disorders. Common lower urinary tract disorders include neurogenic and non-neurogenic overactive bladder, interstitial cystitis, prostatitis, prostadynia, and benign prostatic hyperplasia. These disorders are frequently associated with urinary incontinence, which may include urge incontinence, stress incontinence, overflow incontinence, functional incontinence, neurogenic incontinence and post-prostatectomy incontinence, urinary urgency, nocturia, and enuresis. Urinary incontinence conditions can also result from Parkinson's disease, multiple sclerosis, muscle disease, muscle weakness, diabetes, spinal cord injury, nerve disorders of the pelvic floor, destruction of the sensory nerve fibers, congenital defects, sphincter damage from trauma or surgery, obesity, urinary tract infections, bladder stones, hormonal imbalances, medications, and blockage of the urethra (e.g., due to an enlarged prostate or kidney stones).

Disorders of the lower urinary tract affect the quality of life of more than 50 million people in the United States every year. Among these disorders, overactive bladder alone is a chronic condition that affects an estimated 17 to 20 million people in the United States. Overactive bladder is typically caused by overactivity of the detrusor muscle, which cause the bladder to contract prematurely. Symptoms of overactive bladder can include urinary frequency, urinary urgency, urinary urge incontinence, nocturia and enuresis. Overactive bladder can involve both peripheral and central control defects, including hypersensitivity of sensory neurons of the bladder (e.g., arising from inflammatory conditions, hormonal imbalances, or prostatic hypertrophy), destruction of the sensory nerve fibers, and damage to the spinal cord or brain stem causing interruption of transmitted signals. Neurogenic overactive bladder (or neurogenic bladder) is caused by detrusor hyperreflexia secondary to neurologic disorders such as stroke, Parkinson's disease, diabetes, multiple sclerosis, peripheral neuropathy, or spinal cord injury. Non-neurogenic overactive bladder is caused by detrusor muscle instability, arising from non-neurological abnormalities such as bladder stones, muscle disease, urinary tract infection and pharmacological side effects.

Current treatments for overactive bladder include medication, diet modification, bladder training, electrical stimulation, and surgery. The most widely used drug treatment employs antimuscarinic agents, such as oxybutynin. However, antimuscarinics have limited efficacy and lack selectivity for the bladder, resulting in numerous side effects such as dry mouth, dry eyes, dry vagina, blurred vision, palpitations, arrhythmia, drowsiness, urinary retention, weight gain, hypertension and constipation.

Interstitial cystitis is a chronic, often severe inflammation of the bladder wall, the cause of which is unknown. This condition predominantly affects young and middle-aged females, although men and children can also be affected. Symptoms of interstitial cystitis can include irritative voiding symptoms, urinary frequency, urinary urgency, nocturia or suprapubic or pelvic pain related associated with voiding.

Currently the only approved medication for use in interstitial cystitis is pentosan polysulfate sodium, which is thought to work by restoring a damaged, thin or leaky bladder surface. However, pentosan polysulfate sodium is not effective in a large percentage of patients, and must be taken continually for several months to yield improvements. Other medications such as antidepressants, antihistamines and anticonvulsants have also been used to treat interstitial cystitis, with limited success.

Prostatitis and prostadynia affect approximately 2-9% of the adult male population. Prostatitis involves inflammation of the prostate, and includes bacterial prostatitis (acute and chronic) and non-bacterial prostatitis. Acute and chronic bacterial prostatitis are characterized by inflammation of the prostate associated with pain, urinary frequency and/or urinary urgency. Chronic bacterial prostatitis is distinguished from acute bacterial prostatitis based on the recurrent nature of the disorder. Chronic non-bacterial prostatitis is characterized by inflammation of the prostate through unknown etiology, with an excessive amount of inflammatory cells in prostatic secretions, and is usually associated with pain, urinary frequency and/or urinary urgency. Prostadynia mimics the symptoms of prostatitis without inflammation of the prostate, bacterial infection of the prostate, or elevated levels inflammatory cells in prostatic secretions. Prostadynia is also commonly associated with pain, urinary frequency and/or urinary urgency.

Currently there are no widely accepted treatments for prostatitis and prostadynia. Antibiotics are often prescribed, but with little evidence of efficacy. COX-2 selective inhibitors and α-adrenergic blockers and have been suggested as treatments, but their efficacy has not been established. Anticholinergic drugs have been employed with limited success in terms of symptomatic relief.

Benign prostatic hyperplasia (BPH) is a disorder associated with enlargement of the prostate gland accompanied by urinary frequency, urinary urgency, urge incontinence, nocturia, and/or reduced urinary force and speed of flow. BPH is usually treated with androgen deprivation therapy, 5α-reductase inhibitors, α-adrenergic blockers, or surgery. These treatments have proven only minimally or moderately effective.

Despite pharmacological advances, there are still no satisfactory treatments for urinary incontinence and associated conditions caused by disorders of the lower urinary tract. Accordingly, there remains an important, unmet need for alternative compositions and methods to treat urinary incontinence and associated conditions caused by neurogenic and non-neurogenic overactive bladder, interstitial cystitis, prostatitis, prostadynia, benign prostatic hyperplasia, and other lower urinary tract disorders in mammalian subjects.

### SUMMARY OF THE DISCLOSURE

It is therefore an object of the present invention to provide novel and improved compositions and methods for treating and managing lower urinary tract disorders in mammalian subjects, including humans.

It is a further object of the invention to provide compositions and methods for treating and preventing symptoms of a lower urinary tract disorder including, but not limited to, urinary incontinence such as urge incontinence, stress incontinence, overflow incontinence, functional incontinence, neurogenic incontinence and post-prostatectomy incontinence, urinary urgency, urinary frequency, nocturia, and enuresis in mammalian subjects.

The invention achieves these objects and satisfies additional objects and advantages by providing methods and compositions for treating and/or preventing urinary incontinence in mammalian subjects using bicifadine.

Useful bicifadine compounds within the formulations and methods of the invention include compounds in the class of 1-phenyl-3-azabicyclo[3.1.0]hexanes having at least one substituent on the phenyl ring and possessing anti-incontinence activity. Useful forms of bicifadine for use herein include various pharmaceutically acceptable active salts, isomers, enantiomers, polymorphs, solvates, hydrates, and/or prodrugs of bicifadine, or combinations thereof. In exemplary embodiments, the compositions and methods of the invention may employ a bicifadine HCl compound to treat symptoms of lower urinary tract disorders, including urinary incontinence.

Mammalian subjects amenable for treatment according to the methods of the invention include, but are not limited to, subjects with overactive bladder including neurogenic and non-neurogenic overactive bladder, interstitial cystitis, prostatitis, prostadynia, and benign prostatic hyperplasia. These disorders of the lower urinary tract may be secondary to Parkinson's disease, multiple sclerosis, muscle disease, or diabetes, or may arise from spinal cord injury, nerve disorders of the pelvic floor, congenital defects such as short urethra, damage to the sphincter from surgery or trauma, obesity, urinary tract infections, bladder stones, hormonal imbalances, destruction of sensory nerve fibers, inflammatory conditions, medication, muscle weakness, or urethral blockage. Additional subjects for treatment according to the invention may exhibit one or more symptoms of urinary incontinence triggered by activity, such as exercise, coughing, laughing or lifting.

These and other subjects are effectively treated, prophylactically and/or therapeutically, by administering to the subject an anti-incontinence effective amount of bicifadine sufficient to prevent or reduce incontinence, or one or more associated condition(s) in the subject. The therapeutically useful methods and formulations of the invention may employ bicifadine in a variety of forms, including its pharmaceutically acceptable salts, isomers, enantiomers, polymorphs, solvates, hydrates, prodrugs, and/or combinations thereof, including an exemplary form of bicifadine, bicifadine HCl, as used in the examples herein for illustrative purposes.

Within additional aspects of the invention, combinatorial formulations and methods are provided which employ an effective amount of bicifadine and one or more additional, adjunctive active agent(s) that is/are combinatorially formulated or coordinately administered with bicifadine to yield an anti-incontinence composition or coordinate treatment response. Exemplary combinatorial formulations and coordinate treatment methods in this context employ bicifadine in combination with one or more additional, adjunctive anti-incontinence agent(s) or other adjunctive therapeutic agents. The adjunctive therapeutic agents used in combination with bicifadine in these embodiments may possess anti-incontincence activity, directly or indirectly, alone or in combination with bicifadine, or may exhibit other useful adjunctive therapeutic activity in combination with bicifadine. Useful adjunctive therapeutic agents in these combinatorial formulations and coordinate treatment methods include, for example, α2δ subunit calcium channel modulators, 4-phenyl substituted tetrahydroisoquinolines, 5-HT3 receptor antagonists, 5-α reductase inhibitors, antibiotics, anticholinergic drugs, anticonvulsants, antidepressants, antihistamines, antimuscarinics, antispasmodics, buprenorphine, calcium antagonists, COX (cyclooxygenase)-2 inhibitors, dibenzazepines, hormones, hydantoins, muscle relaxants, noradrenaline reuptake inhibitors, NSAIDS, parasympatholytics, potassium channel openers, prostaglandin synthesis inhibitors, sodium channel modulators, vasopressin analogues, α-adrenoreceptor antagonists, and β-adrenoreceptor agonists. Yet additional coordinate treatment methods in this context employ bicifadine treatment in combination with one or more adjunctive therapies including, but not limited to, diet modification, bladder training, muscle training, biofeedback, behavioral modification, bladder reflex training, and electrical stimulation.

### BRIEF DESCRIPTION OF THE DRAWINGS

FIG. 1 is a graph depicting the normalization of bladder capacity in a feline test model in which the subject was treated with bicifadine following acetic acid challenge.

FIG. 2 is a graph depicting the effects of bicifadine on latency (time) to bladder contraction in a feline test model in which the subject was treated with bicifadine following acetic acid challenge.

FIG. 3 is a graph depicting the normalization of bladder capacity in a feline test model in which the subject was treated with (+)-bicifadine following acetic acid challenge.

FIG. 4 is a graph depicting the effects of (+)-bicifadine on latency (time) to bladder contraction in a feline test model subjects treated with (+)-bicifadine following acetic acid challenge

### DETAILED DESCRIPTION OF EXEMPLARY EMBODIMENTS

The instant invention provides novel methods and compositions for preventing and/or treating lower urinary tract disorders in mammalian subjects. In various embodiments, the methods and compositions are effective to prevent or treat symptoms of a lower urinary tract disorder including, but not limited to, urinary incontinence. As used herein, the term “urinary incontinence” is intended to include a range of conditions including urge incontinence, stress incontinence, overflow incontinence, functional incontinence, neurogenic incontinence, post-prostatectomy incontinence, urinary frequency, urinary urgency, nocturia, enuresis, and related conditions in mammalian subjects. In more detailed embodiments, the lower urinary tract disorder, or targeted symptoms for treatment arising therefrom, may include overactive bladder, including neurogenic and non-neurogenic overactive bladder, interstitial cystitis, prostatitis, prostadynia, and benign prostatic hyperplasia. In further embodiments, the methods and compositions of the invention are effective for preventing or treating excessive micturition in subjects suffering from lower urinary tract disorders.

Anti-incontinence formulations and methods provided herein employ bicifadine as a novel anti-incontinence agent. Within these formulations and methods, the bicifadine may be provided in any of a variety of forms, including any pharmaceutically acceptable, active salt, isomer, enantiomer, solvate, hydrate, polymorph or prodrug of bicifadine, and/or combinations thereof. In exemplary compositions and methods, bicifadine HCl is effectively used to treat urinary incontinence in mammalian subjects suffering from lower urinary tract disorders, including, but not limited to, subjects suffering from overactive bladder, including neurogenic and non-neurogenic overactive bladder, interstitial cystitis, prostatitis, prostadynia, and benign prostate hyperplasia.

A broad range of mammalian subjects, including human subjects, are amenable to treatment using the formulations and methods of the invention. These subjects include, but are not limited to, human and other mammalian subjects presenting with a condition of urinary incontinence, such as urge incontinence, stress incontinence, overflow incontinence, functional incontinence, neurogenic incontinence, post-prostatectomy incontinence, urinary frequency, urinary urgency, nocturia, enuresis, as well as subjects presenting with related disorders of the lower urinary tract including, but not limited to, subjects suffering from overactive bladder, interstitial cystitis, prostatitis, prostadynia, and benign prostate hyperplasia.

Within the methods and compositions of the invention, bicifadine is effectively formulated or administered as an anti-incontinence agent effective for treating urinary incontinence and/or related disorders of the lower urinary tract in mammals. In exemplary embodiments, bicifadine HCl is shown to be an anti-incontinence effective agent in pharmaceutical formulations and therapeutic methods, alone or in combination with one or more adjunctive therapeutic agent(s). The present disclosure further provides additional, pharmaceutically acceptable bicifadine compounds, complexes, derivatives, salts, solvates, isomers, enantiomers, polymorphs, and prodrugs, and combinations thereof, which are effective as anti-incontinence therapeutic agents within the methods and compositions of the invention.

Anti-incontinence compositions, including pharmaceutical formulations of the invention, comprise an anti-incontinence effective amount of bicifadine, which is effective for prophylaxis and/or treatment of urinary incontinence. Typically, an anti-incontinence effective amount of bicifadine will comprise an amount of the active drug which is therapeutically effective, in a single or multiple unit dosage form, over a specified period of therapeutic intervention, to measurably alleviate one or more symptoms of a lower urinary tract disorder in the subject, including but not-limited to urge incontinence, stress incontinence, overflow incontinence, functional incontinence, neurogenic incontinence, post-prostatectomy incontinence urinary frequency, urinary urgency, nocturia, and enuresis. Within exemplary embodiments, these compositions are effective within in vivo treatment methods to alleviate urinary incontinence, urinary urgency, nocturia, and enuresis associated with neurogenic and non-neurogenic overactive bladder, interstitial cystitis, prostatitis, prostadynia, and benign prostatic hyperplasia, Parkinson's disease, multiple sclerosis, muscle disease or debility, diabetes, spinal cord injury, nerve disorders of the pelvic floor, congenital defects, damage to the sphincter from surgery or trauma, obesity, urinary tract infections, bladder stones, hormonal imbalances, destruction of the sensory nerve fibers, inflammatory conditions, medications, and blocked urethra (e.g., due to enlargement of the prostate or kidney stones).

Anti-incontinence compositions of the invention typically comprise an anti-incontinence effective amount or unit dosage of bicifadine, which may be formulated with one or more pharmaceutically acceptable carriers, excipients, vehicles, emulsifiers, stabilizers, preservatives, buffers, and/or other additives that may enhance stability, delivery, absorption, half-life, efficacy, pharmacokinetics, and/or pharmacodynamics, reduce adverse side effects, or provide other advantages for pharmaceutical use. Anti-incontinence effective amounts of bicifadine (e.g., a unit dose or concentration of bicifadine HCl, or of any selected pharmaceutically acceptable salt(s), isomer(s), enantiomer(s), solvate(s), polymorph(s) and/or prodrug(s) of bicifadine) will be readily determined by those of ordinary skill in the art, depending on clinical and patient-specific factors. Suitable effective unit dosage amounts for mammalian subjects, including humans, may range from 25 to 1800 mg, 50 to 1000 mg, 75 to 900 mg, 100 to 750 mg, or 150 to 500 mg. In certain embodiments, the anti-incontinence effective dosage of bicifadine may be selected within narrower ranges of, for example, 10 to 25 mg, 30-50 mg, 75 to 100 mg, 100 to 250 mg, or 250 to 500 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In one exemplary embodiment, dosages of 10 to 25 mg, 30-50 mg, 75 to 100 mg, 100 to 250 mg, or 250 to 500 mg, are administered one, two, three, or four times per day. In more detailed embodiments, dosages of 50-75 mg, 100-200 mg, 250-400 mg, or 400-600 mg are administered once or twice daily. In alternate embodiments, dosages are calculated based on body weight, and may be administered, for example, in amounts from about 0.5 mg/kg to about 20 mg/kg per day, 1 mg/kg to about 15 mg/kg per day, 1 mg/kg to about 10 mg/kg per day, 2 mg/kg to about 20 mg/kg per day, 2 mg/kg to about 10 mg/kg per day or 3 mg/kg to about 15 mg/kg per day.

The amount, timing and mode of delivery of compositions of the invention comprising an anti-incontinence effective amount of bicifadine will be routinely adjusted on an individual basis, depending on such factors as weight, age, gender, and condition of the individual, the acuteness of the incontinence and/or related symptoms, whether the administration is prophylactic or therapeutic, and on the basis of other factors known to effect drug delivery, absorption, pharmacokinetics, including half-life, and efficacy.

An effective dose or multi-dose treatment regimen for the instant anti-incontinence formulations will ordinarily be selected to approximate a minimal dosing regimen that is necessary and sufficient to substantially prevent or alleviate urinary incontinence in the subject, and/or to substantially prevent or alleviate one or more symptoms associated with a lower urinary tract disorder in the subject. In the case of urinary incontinence associated with infection, for example, a dosage and administration protocol will often include repeated dosing therapy over a course of several days or even one or more weeks. In the case of urinary incontinence secondary to disease or injury, an effective treatment regime may involve prophylactic dosage administered on a day or multi-dose per day basis lasting over the course of days, weeks, months or even years. In acute cases, the effective dose of bicifadine may comprise, for example, 50 to 800 mg per day, given in one or multiple intravenous bolus injections or by infusion.

Various assays and model systems can be readily employed to determine the therapeutic effectiveness of anti-incontinence bicifadine treatment according to the invention. Efficacy in this context may be demonstrated, for example, by a decrease in urinary frequency in a subject suffering from a lower urinary tract disorder, e.g., to lower urinary frequency from about ten-twelve or more times a day, to about eight or more times a day, or to about six times a day, or to about four or five times a day.

Therapeutic efficacy of bicifadine treatment according to the invention may be alternatively demonstrated in certain subjects by a change in results of electromyography (EMG) and nerve conduction studies. In an abnormal EMG of a subject suffering from a lower urinary tract disorder, spontaneous electrical activity can be detected in a muscle associated with urinary control in a resting state. The speed of nerve impulse transmission may be slower or faster than normal for that nerve as an indicator of the subject urological disorder. Following treatment according to the compositions and methods herein, EMG results in a subject suffering from a lower urinary tract disorder will normalize, approaching a normal value or become normal (e.g., showing no electrical activity when the muscle is at rest, smooth wavelike forms with each muscle contraction, and involving transmission of electrical impulses at approximately normal speeds).

Therapeutic effectiveness of bicifadine treatment according to the invention may alternatively be demonstrated, for example, through cystometry. In exemplary embodiments, anti-incontinence efficacy of the compositions and methods of the invention will be associated with an urge to urinate in the treated subject when the bladder contains at least about 150 ml to about 200 ml of urine, often when the bladder has filled to at least about 150 ml to about 300 ml of urine, and in many cases when bladder content reaches about 200 ml to 500 ml of urine.

A reduction in stress incontinence may also indicate therapeutic effectiveness of bicifadine treatment. In a stress incontinence test, the bladder of a subject is filled with water or saline and the subject is then asked to cough, bend over, or lift a heavy object. Stress incontinence is demonstrated by involuntary leakage of urine. Effectiveness of bicifadine treatment may be demonstrated in a reduction of leakage, a 10%, 20%, 30%, 50% or greater reduction, up to a 75-90%, or 95% or greater, reduction, in leakage, compared to placebo-treated or other suitable control subjects.

Therapeutic effectiveness of bicifadine treatment according to the invention will often be determined by a decrease in post-void residual amounts in the bladder, for example to values of from about 200 ml to about 150 ml, 100 ml, 75 ml, 50 ml, or 25 ml. Therapeutic effectiveness may alternatively be demonstrated by a decrease in bladder voiding time of 100 ml, for example to a decreased time of about 50 seconds, 30 seconds, 20 seconds, or 10 seconds. Anti-incontinence efficacy may further be demonstrated by a decrease in bladder voiding time of 400 ml, e.g., to yield an improved voiding time of about 50 seconds, 30 seconds, 25 seconds, to less than 20 seconds.

Anti-incontinence effectiveness of the methods and compositions of the invention can be alternatively demonstrated in certain subjects by a decrease in any one or assemblage of symptoms caused by, or associated with, lower urinary tract disorders in mammalian subjects, including urinary incontinence. For each of the indicated conditions described herein, test subjects will exhibit a 10%, 20%, 30%, 50% or greater reduction, up to a 75-90%, or 95% or greater, reduction, in one or more symptoms caused by, or associated with, a lower urinary tract disorder in the subject, compared to placebo-treated or other suitable control subjects.

Within other exemplary embodiments of the invention, bicifadine formulations and methods are provided for effective management, prophylaxis, and/or treatment of overactive bladder. In subjects suffering from overactive bladder, the detrusor muscle contracts spastically, sometimes without a known cause, which results in sustained, high bladder pressure, urge incontinence, urgency, nocturia and/or enuresis. While the cause of overactive bladder is unknown, it can be associated with inflammatory conditions, hormonal imbalance, prostate hypertrophy, destruction of the sensory nerve fibers, damage to the spinal cord or brain stem, bladder disease, infection, Parkinson's disease, multiple sclerosis and peripheral neuropathy. In a normal bladder, the detrusor muscle contracts and relaxes in response to the volume of urine in the bladder and the initiation of urination. However, subjects with overactive bladder often experience urgency at inconvenient and unpredictable times and sometimes lose control. Thus, overactive bladder often interferes with work, daily routine, social interactions, intimacy and sexual function, often yielding profound adverse psychological impacts.

The methods and formulations of the invention for treating overactive bladder employ an effective amount of bicifadine in a pharmaceutical composition suitable for administration to mammalian subjects. The methods and formulations deliver an effective amount of bicifadine to prevent, or substantially alleviate, one or more of the above-identified adverse symptoms associated with overactive bladder. Thus, following administration of the inventive bicifadine formulation or method, subjects will exhibit a 10%, 20%, 30%, 50% or greater reduction, up to a 75-90% reduction, in one or symptoms associated with overactive bladder, compared to placebo-treated or other suitable control subjects.

Within additional exemplary embodiments of the invention, bicifadine formulations and methods are provided for effective management, prophylaxis, and/or treatment of interstitial cystitis. Interstitial cystitis is a chronic inflammatory condition of the bladder that causes urinary frequency, urgency, nocturia, irritative voiding symptoms, and pelvic discomfort. In subjects with interstitial cystitis, the glycosaminoglycan layer that normally protects the bladder epithelium breaks down, allowing toxins to irritate the bladder wall, which becomes inflamed, decreasing its capacity to store urine. Although no bacteria or viruses have been found in the urine of interstitial cystitis sufferers, an unidentified infectious agent may be the cause. Other reports associate interstitial cystitis with autoimmune disease, asthma, endometriosis, food allergies, pollen allergy, irritable bowel syndrome, lupus, migraine, rheumatoid arthritis, and sinusitis.

By administering the anti-incontinence bicifadine formulations of the invention in a suitable prophylactic or therapeutic treatment protocol, subjects presenting with, or at an elevated risk for, interstitial cystitis can be effectively treated. Treatment of these conditions using the formulations and methods provided herein will reduce or prevent urinary incontinence in these subjects, and will often additionally substantially prevent or alleviate one or more of the above-identified symptoms associated with interstitial cystitis. Treatment of associated conditions in this context may involve the use of combinatorial bicifadine formulations or coordinated treatment methods, combining bicifadine with one or more adjunctive therapeutic agents, for example one or more adjunctive, anti-incontinence agents, antibiotics, analgesics, etc.

Within yet additional exemplary embodiments of the invention, bicifadine formulations and methods are provided for effective management, prophylaxis, and/or treatment of prostatitis. Prostatitis is inflammation of the prostate gland. While not wishing to be bound, it is currently theorized that prostatitis is generally caused by bacterial infection, but evidence of infection is not always found. An infected or inflamed prostate can cause painful urination and ejaculation, and if left untreated, serious complications including fatality can result. Acute bacterial prostatitis is inflammation of the prostate gland caused by bacteria such as Escherichia coli and the Klebsiella species. Chronic bacterial prostatitis is a recurrent infection and inflammation of the prostate and urinary tract. Nonbacterial prostatitis is an inflamed prostate without bacterial infection. Prostadynia, sometimes called chronic pelvic pain syndrome, is the occurrence of prostatitis symptoms, without inflammation or bacterial infection.

By administering the anti-incontinence bicifadine formulations of the invention in a suitable prophylactic or therapeutic treatment protocol, subjects presenting with, or at an elevated risk for all forms of prostatitis can be effectively treated. Treatment of these conditions using the formulations and methods provided herein will reduce or prevent urinary incontinence in these subjects, and will often additionally substantially prevent or alleviate one or more of the above-identified symptoms associated with prostatitis as well. Treatment of associated conditions in this context may involve the use of combinatorial bicifadine formulations or coordinated treatment methods, combining bicifadine with one or more adjunctive therapeutic agents, for example one or more adjunctive, anti-incontinence agents, antibiotics, analgesics, etc.

As noted above, the anti-incontinence compositions and methods of the invention are useful to treat or prevent various forms of incontinence, along with related conditions associated with a lower urinary tract disorder. These compositions and methods are, for example, effective to alleviate or prevent urge incontinence, stress incontinence, overflow incontinence, functional incontinence, neurogenic incontinence, post-prostatectomy incontinence, nocturia and enuresis. Urge Incontinence is the inability to prevent urinary leakage when feeling a strong urge to urinate. Symptoms include frequent urination, voiding small amounts of urine, strong urge to urinate, and an inability to get to the bathroom prior to leakage. Stress Incontinence is the loss of urine when sneezing, coughing, laughing, lifting or doing strenuous activity such as exercising. Overflow Incontinence occurs when the bladder does not empty properly and at a certain volume begins to overflow, causing leakage. Symptoms include a palpably swollen bladder, supra-pubic tenderness, and reduced urine stream. Functional Incontinence is the inability of the subject to get to the bathroom in time. Nocturia is the frequent need to urinate during the night. Enuresis is the inability to maintain urinary control during sleep, often known as bed wetting. By administering the anti-incontinence bicifadine formulations of the invention in a suitable prophylactic or therapeutic treatment protocol, subjects presenting with, or at an elevated risk for, all forms of urinary incontinence can be effectively treated.

Within additional aspects of the invention, combinatorial anti-incontinence formulations and coordinate administration methods are provided which employ an effective amount of bicifadine and one or more additional active agent(s) that is/are combinatorially formulated or coordinately administered with bicifadine to yield an anti-incontinence composition or coordinate treatment method. Exemplary combinatorial formulations and coordinate treatment methods in this context employ bicifadine in combination with one or more additional anti-incontinence agents, or with another, adjunctive therapeutic agent (e.g., an antibiotic, hormone, analgesic, anxiolytic, or antidepressant agent), and/or in combination with one or more additional therapies. For these combinatorial formulations and coordinate treatment methods, bicifadine is formulated, or coordinately administered, in combination with one or more secondary therapeutic agent(s), which will often be combinatorially effective or coordinately useful to treat symptoms associated with a lower urinary tract disorder in the subject. Exemplary combinatorial formulations and coordinate treatment methods in this context employ bicifadine in combination with one or more additional therapeutic agents selected from anticholinergic drugs, COX-2 inhibitors, antibiotics, antimuscarinics, antidepressants, antihistamines, anticonvulsants, 5-α reductase inhibitors, a adrenoreceptor agonists, β adrenoreceptor agonists, prostaglandin synthesis inhibitors, vasopressin analogues, calcium channel blockers, and potassium channel openers. In other exemplary embodiments, bicifadine will be administered coordinately with one or more additional therapies, for example, diet modification, pelvic floor training, muscle awareness, muscle training, biofeedback, behavioral modification, bladder reflex triggering, bladder training, and electrical stimulation.

In certain embodiments the invention provides combinatorial anti-incontinence formulations comprising bicifadine and one or more adjunctive agent(s) having anti-incontinence activity. Within such combinatorial formulations, bicifadine and the adjunctive agent(s) having anti-incontinence activity will be present in a combined formulation in anti-incontinence effective amounts, alone or in combination. In exemplary embodiments, bicifadine and a non-bicifadine anti-incontinence agent will each be present in an anti-incontinence amount (i.e., in singular dosage which will alone elicit a detectable anti-incontinence response in the subject). Alternatively, the combinatorial formulation may comprise one or both of the bicifadine and non-bicifadine agents in sub-therapeutic singular dosage amount(s), wherein the combinatorial formulation comprising both agents features a combined dosage of both agents that is collectively effective in eliciting an anti-incontinence response. Thus, one or both of the bicifadine and non-bicifadine agents may be present in the formulation, or administered in a coordinate administration protocol, at a sub-therapeutic dose, but collectively in the formulation or method they elicit a detectable anti-incontinence response in the subject.

Exemplary non-bicifadine anti-incontinence agents for use within these aspects of the invention include, but are not limited to, α2δ subunit calcium channel modulator s, 4-phenyl substituted tetrahydroisoquinolines, 5-HT3 receptor antagonists, 5-α reductase inhibitors, antibiotics, anticholinergic drugs, anticonvulsants, antidepressants, antihistamines, antimuscarinics, antispasmodics, buprenorphine, calcium antagonists, COX-2 inhibitors, dibenzazepines, hormones, hydantoins, muscle relaxants, noradrenaline reuptake inhibitors, NSAIDS, parasympatholytics, potassium channel openers, prostaglandin synthesis inhibitors, sodium channel modulators, vasopressin analogues, α-adrenoreceptor antagonists, and β-adrenoreceptor agonists.

To practice the coordinate administration methods of the invention, bicifadine is administered, simultaneously or sequentially, in a coordinate treatment protocol with one or more of the secondary therapeutic agents contemplated herein. Thus, in certain embodiments bicifadine is administered coordinately with a non-bicifadine anti-incontinence agent, or any other secondary therapeutic agent contemplated herein, using separate formulations or a combinatorial formulation as described above (i.e., comprising both the bicifadine and non-bicifadine therapeutic agent). This coordinate administration may be done simultaneously or sequentially in either order, and there may be a time period while only one or both (or all) active therapeutic agents individually and/or collectively exert their biological activities. A distinguishing aspect of all such coordinate treatment methods is that the bicifadine exerts at least some detectable anti-incontinence activity, which yields a favorable clinical response in conjunction with a complementary anti-incontinence, or distinct, clinical response provided by the secondary therapeutic agent. Often, the coordinate administration of bicifadine with the adjunctive therapeutic agent will yield improved continence in the subject beyond any anti-incontinence effects elicited by the secondary therapeutic agent. This qualification contemplates both direct effects, as well as indirect effects (e.g., such as reduced incontinence associated with coordinate administration of an antibiotic, which indirectly reduces incontinence by resolving an incontinence-inducing bacterial condition)

Within exemplary embodiments, bicifadine will be coordinately administered (simultaneously or sequentially, in combined or separate formulation(s)) with one or more secondary anti-incontinence inducing agents, or other therapeutic agents, e.g., selected from α2δ subunit calcium channel modulators, 4-phenyl substituted tetrahydroisoquinolines, 5-HT3 receptor antagonists, 5-α reductase inhibitors, antibiotics, anticholinergic drugs, anticonvulsants, antidepressants, antihistamines, antimuscarinics, antispasmodics, bradykinin receptor agonists, buprenorphine, calcium antagonists, COX-2 inhibitors, dibenzazepines, hormones, hydantoins, muscle relaxants, neurokinin receptor agonists, noradrenaline reuptake inhibitors, nitric oxide donors, NSAIDS, parasympatholytics, potassium channel openers, prostaglandin synthesis inhibitors, sodium channel modulators, vasopressin analogues, α-adrenoreceptor antagonists, and β-adrenoreceptor agonists. Exemplary anti-incontinence α2δ subunit calcium channel modulators include, but are not limited to gamma-aminobutyric acid analogs such as gabapentin and pregablin. Exemplary anti-incontinence anticholinergic drugs in this context include, but are not limited to, oxybutin chloride, oxybutynin, tolterodine tartrate, flavoxate hydrochloride, hyoscyamine sulfate, scopolamine butylbromide, trospium chloride, darifenacin, propiverine, dicyclomine hydrochloride, arenzipine, methoctramine, tropicamide or propantheline. Exemplary anti-incontinence anticonvulsants include, but are not limited to, losigamore, zonisamide, topiramate, rufinamide, harkoseride, memantine hydrochloride, felbamate, or valproate. Exemplary anti-incontinence anti-depressants include tricyclic antidepressants, imipramine, amitriptyline, or duloxetine. Exemplary anti-incontinence antihistamines include, but are not limited to, loratidine, or chlorpheniramine. Exemplary anti-incontinence antimuscarinics in this context include, but are not limited to, oxybutynin, tolterodine, propiverine, trospium, solifenacin, darifenacin, propiverine, propantheline bromide, hyoscyamine sulfate, dicyclomine hydrochloride, flavoxate hydrochloride, pirenzipine, methoctramine, atropine or tropicamide. Exemplary anti-incontinence calcium antagonists include, but are not limited to verapamil or nifedipine. Exemplary anti-incontinence COX-2 inhibitors in this context include, but are not limited to, nitroflurbiprofen, rofecoxib, or celecoxib. Exemplary anti-incontinence dibenzazepines in this context include, but are not limited to, carbamazepine, oxcarbazepine, or licarbazepine. Exemplary anti-incontinence hydantoins in this context include, but are not limited to, phenytoin sodium, or fosphenytoin sodium. Exemplary anti-incontinence muscle relaxants include, but are not limited to flavoxate. Exemplary anti-incontinence noradrenaline reuptake inhibitors include, but are not limited to, reboxetine, lefepramine, desipramine, nortriptyline, maprotiline, oxaprotiline, levoprotiline, viloxazine, and atomoxetine. Exemplary anti-incontinence parasympatholytics includine, but are not limited to, oxybutynine, propiverine or tolterodine. Exemplary anti-incontinence prostaglandin synthesis inhibitors include, but are not limited to indomethacin, or flurbiprofen. Exemplary anti-incontinence anti-spasmodics in this context include, but are not limited to, alibendol, ambucetamide, aminopromazine, apoatropine, bevonium methyl sulfate, bietamiverine, butaverine, butropium bromide, n-butylscopolammonium bromide, caroverine, cimetropium bromide, cinnamedrine, clebopride, coniine hydrobromide, coniine hydrochloride, cyclonium iodide, difemerine, diisopromine, dioxaphetyl butyrate, diponium bromide, drofenine, emepronium bromide, ethaverine, feclemine, fenalamide, fenoverine, fenpiprane, fenpiverinium bromide, fentonium bromide, flavoxate, flopropione, gluconic acid, guaiactamine, hydramitrazine, hymecromone, leiopyrrole, mebeverine, moxaverine, nafiverine, octamylamine, octaverine, pentapiperide, phenamacide hydrochloride, phloroglucinol, pinaverium bromide, piperilate, pipoxolanhydrochloride, pramiverin, prifinium bromide, properidine, propivane, propyromazine, prozapine, racefemine, rociverine, spasmolytol, stilonium iodide, sultroponium, tiemonium iodide, tiquizium bromide, tiropramide, trepibutone, tricromyl, trifolium, trimebutine, n,n-1trimethyl-3,3-diphenyl-propylamine, tropenzile, trospium chloride, or xenytropium bromide. Exemplary anti-incontinence sodium channel modulators include, but are not limited to, ralfinamide, aryldiazines, aryltriazines, lamotrigine carbamazepine, phenytoin sodium, fosphenytoin sodium, tocainide, flecainide, benzamide monoacetate, mexiletine hydrochloride ropivacaine hydrochloride lidocaine, acetamide, mepivacaine, bupivacaine, etidocaine, tetracaine, dibucaine, or soretolide. Exemplary anti-incontinence vasopressin analogs include, but are not limited to, desmopressin. Exemplary anti-incontinence α-adrenoreceptor antagonists include, but are not limited to, alfuzosin, doxazosin, prazosin, terazosin, pseudoephedrine or tamsulosin. Exemplary anti-incontinence β-adrenoreceptor agonists include, but are not limited to, terbutaline, pindolol clenbuterol, or sambutanol. Other useful anti-incontinence agents include, but are not limited to, baclofen, capsaicin, and resiniferatoxin.

These combinatorial formulations may also be used in conjunction with one or more additional therapies including, but not limited to, diet modification, bladder training, pelvic floor training, muscle awareness, muscle training, biofeedback, bladder training behavioral modification, bladder reflex triggering, electrical stimulation and surgery.

As noted above, in all of the various embodiments of the invention contemplated herein, the anti-incontinence methods and formulations may employ bicifadine in a variety of forms, including any one or combination of its pharmaceutically acceptable salts, isomers, enantiomers, polymorphs, solvates, hydrates, and/or prodrugs. In exemplary embodiments of the invention, bicifadine hydrochloride is employed within the therapeutic formulations and methods for illustrative purposes.

Bicifadine HCl, ((±)-1-(4-methylphenyl)-3-azabicyclo[3.1.0]hexane hydrochloride. DOV 220,075), also named racemic 1-(p-toyl)-3-azabicyclo[3.1.0]hexane hydrochloride, is a non-narcotic analgesic disclosed in U.S. Pat. No. 4,231,935 and U.S. Pat. No. 4,196,120. It is represented by the structural formula I:

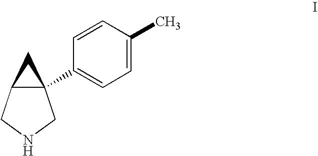

Bicifadine HCl also exists in at least two polymorphic crystalline forms, designated polymorph forms A and B (as described in U.S. patent application Ser. No. 10/702,397, herein incorporated by reference). Other polymorphic forms of bicifadine hydrochloride may exist and are considered to be within this disclosure.

Polymorphs include compounds with identical chemical structure but different internal structures. Additionally, many pharmacologically active organic compounds regularly crystallize incorporating second, foreign molecules, especially solvent molecules, into the crystal structure of the principal pharmacologically active compound forming pseudopolymorphs. When the second molecule is a solvent molecule, the pseudopolymorphs can also be referred to as solvates. All of these additional forms of bicifadine are likewise useful within the anti-incontinence methods and formulations of the invention.

Polymorph form A of bicifadine HCL can be formed by at least any of the methods disclosed in U.S. Pat. No. 4,231,935 and U.S. Pat. No. 4,196,120 (each of which is incorporated herein by reference). Polymorph form B can be formed by any suitable method, including the methods disclosed in U.S. patent application Ser. No. 10/702,397, herein incorporated by reference. For example, polymorph B can be formed from polymorph form A through the application of kinetic energy and through crystallization techniques. In one embodiment, kinetic energy in the form of agitating, stirring, grinding or milling can be applied to polymorph form A especially at low temperatures, generally from about −200° C. to about 50° C., in another embodiment from about −200° C. to about 35° C., in a further embodiment from about −200° C. to about 0° C. In another embodiment, polymorph B can be crystallized from a solution of polymorph A can be heated and allowed to cool for a sufficient amount of time to form polymorph B.

The polymorphs of bicifadine HCl may be characterized by their infrared spectra and/or their x-ray powder diffraction pattern. The X-ray powder diffraction (XRPD) analyses of polymorph forms A and B of racemic bicifadine hydrochloride were performed with a Shimadzu XRD-6000 X-ray powder diffractometer using Cu Ka radiation. The bicifadine was loaded onto the machine as a crystalline powder. The instrument was equipped with a fine focus X-ray tube. The tube voltage and amperage were set to 40 kV and 40 mA, respectively. The divergence and scattering slits were set at 1° and the receiving slit was set at 0.15 mm. Diffracted radiation was detected by a NaI scintillation detector. A theta-two theta continuous scan at 3/min (0.4 sec/0.02°step) from 2.5 to 40°2θ was used. A silicon standard was analyzed to check the instrument alignment. Data were collected and analyzed using XRD-6000 v.4.1.

The X-ray powder diffraction pattern of polymorph form A of racemic bicifadine hydrochloride is given in terms of “d” spacing and relative intensities (I) is as follows (s=strong, m=medium, w=weak, v=very, d=diffuse) and these terms are set forth in Table 1 below, and the X-ray powder diffraction pattern of form B of bicifadine hydrochloride is set forth in Table 2 below:

TABLE 1
Peak Positions, d-Spacings, and Intensities for
Polymorph Form A Bicifadine Hydrochloride

| 2θ (deg) | d (Å) | I^{a} |
|---|---|---|
| 5.35 | 16.50 | Vs |
| 10.61 | 8.33 | Vs |
| 11.45 | 7.72 | W |
| 15.22 | 5.82 | W |
| 15.93 | 5.56 | W |
| 16.97 | 5.22 | W |
| 18.37 | 4.83 | W |
| 20.04 | 4.43 | Md |
| 20.26 | 4.38 | Md |
| 21.22 | 4.18 | M |
| 21.89 | 4.06 | S |
| 23.12 | 3.84 | Md |
| 23.54 | 3.78 | Wd |
| 26.63 | 3.34 | M |
| 27.83 | 3.20 | Wd |
| 28.32 | 3.15 | Wd |
| 30.67 | 2.91 | Wd |
| 32.03 | 2.79 | S |
| 37.57 | 2.39 | W |
| 38.20 | 2.35 | W |

^{a}s = strong, m = medium, w = weak, v = very, d = diffuse

TABLE 2
Peak Positions, d-Spacings, and Intensities for
Polymorph Form B Bicifadine Hydrochloride

| 2θ (deg) | d (Å) | I^{a} |
|---|---|---|
| 5.08 | 17.39 | Vs |
| 10.07 | 8.77 | S |
| 15.19 | 5.83 | S |
| 16.83 | 5.27 | S |
| 18.64 | 4.76 | Md |
| 18.76 | 4.73 | Md |
| 19.64 | 4.52 | W |
| 20.16 | 4.40 | M |
| 21.96 | 4.05 | M |
| 22.37 | 3.97 | S |
| 23.16 | 3.84 | W |
| 24.00 | 3.70 | W |
| 25.27 | 3.52 | D |
| 27.33 | 3.26 | Md |
| 27.74 | 3.21 | M |
| 29.00 | 3.08 | M |
| 30.43 | 2.93 | Md |
| 31.84 | 2.80 | Wd |
| 32.29 | 2.77 | W |
| 35.27 | 2.54 | Wd |
| 35.64 | 2.52 | W |

^{a}s = strong, m = medium, w = weak, v = very, d = diffuse

Table 1 and Table 2 represent the XRPD pattern of the peak positions of bicifadine hydrochloride form A and form B respectively having reduced particle size. The results in these tables demonstrate the difference between the XRPD patterns of form A and form B at a reduced particle size. However, there are key peaks at given angles in this pattern which identify polymorph form B of bicifadine hydrochloride and are typically present in the XRPD pattern of polymorph form B irrespective of its particle size. These angles, expressed as 2θ (deg), locating these major peaks which characterize the polymorph form B, using Cu Ka radiation, are: 5.08; 10.07; 20.16; 25.17; and 30.43.

The infrared spectra were obtained for each of the samples using a Magna-IR 860® Fourier transform infrared (FT-IR) spectrophotometer (Thomas Nicolet) equipped with an Ever-Glo mid/far IR source, an extended range potassium bromide (KBr) beamsplitter, and a deuterated triglycine sulfate (DTGS) detector. The spectrophotometer measured the intensity of infrared light bands of each of the samples at given wavelengths. A diffuse reflectance accessory (the Collector™, Thermo Spectra-Tech) was used for sampling. Each spectrum represents 256 co-added scans collected from 400-4000 cm⁻¹ at a spectral resolution of 4 cm⁻¹. Sample preparation consisted of placing the sample of powder containing crystals in either polymorph form A or form B into a 13-mm diameter cup and leveling the material with a frosted glass slide. A background data set was acquired with an alignment mirror in place. The reflectance R is the ratio, at a give wavenumber, of the light intensity of the sample/light intensity of the background set. A Log 1/R(R=reflectance) spectrum acquired by taking a ratio of these two data sets (the sample and the background light intensities) against each other. The infrared spectrum of polymorph A or racemic bicifadine hydrochloride as a dry crystalline powder, as provided in Table 3, showed the indicated main peaks which characterized this polymorph. The infrared spectrum of polymorph B of racemic bicifadine hydrochloride in dry crystalline powder, as provided in Table 4, showed the indicated main peaks which characterize this polymorph.

TABLE 3
Infrared Peak Positions For Polymorph Form A Bicifadine
Hydrochloride. All values in wavenumbers (cm⁻¹)

3949
2923
2431
2280
2091
1895
1790
1595
1522
1430
1376
1233
1130
1088
1068
1050
900
825
781
714
689
652
574
533
437

TABLE 4
Infrared Peak Positions for Polymorph Form B Bicifadine
Hydrochloride. All values in wavenumbers (cm⁻¹)

3185
2769
2437
2276
2108
1908
1804
1658
1596
1518
1453
1403
1343
1305
1274
1209
1131
1111
1022
963
904
891
856
818
783
719
684
660
637
580
532
475
422

Table 3 and Table 4 provide the complete patterns of the infrared peak positions with respect to polymorph form A and polymorph form B of bicifadine hydrochloride respectively. However, there are certain key peaks, within this pattern, which are associated with polymorph form B of bicifadine hydrochloride and are sufficient to characterize this polymorph. These peaks, expressed in wavenumbers (cm⁻¹), are: 2108; 891; 856; 719; and 660.

Effective dosages of bicifadine may comprise any crystalline polymorphic or amorphous form of the compound, or mixture(s) thereof. For example, the effective dosage of bicifadine in a therapeutic formulation as provided herein may comprise substantially pure bicifadine HCl polymorph “form A”, essentially pure polymorph “form B”, or any mixture of polymorph forms A and B. In certain embodiments, the composition may contain from about 10% to 98% polymorph form B. In other embodiments there may be present in the formulation greateer than about 50% polymorph form B, greater than about 75% polymorph B, or greater than about 90% polymorph B.

Suitable routes of administration for anti-incontinence and related, combinatorial composistions of the invention comprising bicifadine include, but are not limited to, oral, buccal, nasal, aerosol, topical, transdermal, mucosal, injectable, slow release, controlled release, iontophoresis, sonophoresis, and including all other conventional delivery routes, devices and methods. Injectable methods include, but are not limited to, intravenous, intramuscular, intraperitoneal, intraspinal, intrathecal, intracerebroventricular, intraarterial, subcutaneous and intranasal routes.

The compositions of the invention for treating urinary incontinence can further include any one or combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art. These additional formulation additives/agents will often be biologically inactive and can be administered to patients without causing deleterious interactions with the active agent.

If desired, the bicifadine can be administered in a controlled release form by use of such controlled release carriers as a hydrophilic slow release polymer, for example hydroxypropyl methyl cellulose, in an oral unit dosage or other suitable form. Other slow release polymers can be utilized, and these will typically have a viscosity in the range of about 100 cps to about 100,000 cps.

Commonly, the anti-incontinence bicifadine compositions of the invention will be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose dextrose, or other sugars, di basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets, which may be prepared by any conventional method of preparing pharmaceutical oral unit dosage forms can be utilized in preparing oral unit dosage forms. Oral unit dosage forms, such as tablets, may contain one or more conventional additional formulation ingredients, including, but are not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants.

Additional bicifadine compositions of the invention may be prepared and administered in any of a variety of inhalation or nasal delivery forms known in the art. Devices capable of depositing aerosolized bicifadine formulations in the sinus cavity or pulmonary alveoli of a patient include metered dose inhalers, nebulizers, dry powder generators, sprayers, and the like. Suitable formulations for administration, wherein the carrier is a liquid, as for example, a nasal spray or as nasal drops, may include aqueous or oily solutions of bicifadine and any additional active or inactive ingredient(s). Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the compositions in a suitable liquid carrier.

Also provided herein are compositions and methods for topical administration of bicifadine to treat urinary incontinence. Topical compositions may comprise bicifadine and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise bicifadine dissolved or dispersed in a portion of a water or other solvent or liquid to be incorporated in the topical composition or delivery device.

Additional bicifadine formulations are provided for parenteral administration, including aqueous and non-aqueous sterile injection solutions which may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The formulations may be presented in unit-dose or multi-dose containers. Bicifadine anti-incontinence formulations may also include polymers for extended release following parenteral administration. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

In other embodiments, anti-incontinence formulations may comprise bicifadine encapsulated in microcapsules, microparticles, or microspheres, prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions.

The above disclosure generally describes the present invention, which is further exemplified by the following examples. These examples are described solely for purposes of illustration, and are not intended to limit the scope of the invention. Although specific terms and values have been employed herein, such terms and values will likewise be understood as exemplary and non-limiting to the scope of the invention.

The following examples demonstrate by in vitro and in vivo methods that bicifadine HCl ((±)-1-(4-methylphenyl)-3-azabicyclo[3.1.0]hexane hydrochloride) is an effective agent to alleviate or prevent urinary incontinence in mammalian subjects. This novel activity and use may be related to the ability of bicifadine to modulate noradrenergic and serotonergic neurotransmission, by a combination of interactions with α₁ and α₂ adrenergic, and 5-HT_{2A} receptors, as well as by inhibition of norepinephrine re-uptake.

Insights into the potential mechanism by which bicifadine HCl expresses its anti-incontinence action are provided in part by biochemical assays (Table 5, below). Bicifadine HCl is shown to occupy binding sites on both α₁ and α₂ adrenergic receptors. In addition, bicifadine HCl significantly inhibits radioligand binding to the 5-HT_{2A} serotonin receptor.

All three of these receptor subtypes are involved in micturition processes in the central and peripheral nervous system. The interaction of bicifadine HCl with this combination of receptors may contribute to the anti-incontinence efficacy of bicifadine HCl. Moreover, the receptor binding profile of bicifadine is expected to significantly reduce side effects compared to other anti-incontinence inducing drugs.

### EXAMPLE I

### Preparation of 1-(p-tolyl)-3-azabicyclo[3.1.0]hexane hydrochloride

To prepare a useful, exemplary bicifadine agent for use as an anti-incontinence drug, 230 ml of thionyl chloride was added to 120 g of p-tolylacetic acid and the solution was allowed to stand at room temperature for 2 hours, after which it was warmed to 60° C. for 1 hour. To this solution 285 g of N-bromosuccinimide and 10 drops of 48% hydrobromic acid were added and the mixture was refluxed on a 90° C. oil bath for 1 hour. An additional 90 ml of thionyl chloride was then added and refluxing continued for an additional 45 minutes. The resulting mixture was distilled under reduced pressure to remove 250 ml of thionyl chloride, and the residual liquid was poured into 500 ml of cold methanol with stirring and ice cooling over 15 minutes. This solution was evaporated under reduced pressure to give a dark oil which was then dissolved in 100 ml of chloroform. The solution was washed with 500 ml of water, dried over magnesium sulfate and filtered. The filtrate was evaporated under reduced pressure to give a dark oil which was distilled to give 94 g of bromoester as a pale yellow liquid, bp. 115°-120° C. (0.05 mm). The pale yellow liquid was then reacted with methyl acrylate-sodium hydride in ether to give dimethyl cis-1-(p-tolyl)-1,2-cyclopropanedicarboxylate, mp 58°-59° C. Hydrolysis with 1 N potassium hydroxide, followed by acidification with 1N hydrochloric acid, yielded cis-1-(p-tolyl)-1,2-cyclopropanedicarboxylic acid as colorless crystals, mp 188°-190° C. A 5.7 g portion of this diacid and 2.02 g of urea in 200 ml of xylene was refluxed for 22 hours, cooled, diluted with benzene and washed with water. The organic layer was diluted with chloroform, dried, concentrated under reduced pressure, and recrystallized from ethyl acetate and petroleum ether to give 1-(p-tolyl)-1,2-cyclopropanedicarboximide as pale yellow crystals, mp 82°-85° C.

To a mixture of 20.1 g of this imide in 600 ml of benzene was added 160 ml of sodium bis(2-methoxyethoxy)aluminum hydride and the reaction was run, after which excess reagent was decomposed with 160 ml of 10 N sodium hydroxide. The benzene layer was washed with water, dried over magnesium sulfate and filtered. The filtrate was evaporated under reduced pressure to give a dark oil which was dissolved in ether, and then dry hydrogen chloride was bubbled into the solution. The resultant precipitate was collected by filtration and recrystallized from acetonitrile-methanol to give 12.1 g of 1-(p-tolyl)-3-azabicyclo[3.1.0]hexane hydrochloride as pale tan plates, mp 207°-208° C.

### EXAMPLE II

### Preparation of (+)-1-(p-Tolyl)-3-azabicyclo[3.1.0]hexane hydrochloride

An alternative, exemplary bicifadine agent for use as an anti-incontinence drug was prepared as follows. A solution of 94.8 g of racemic-1-(p-tolyl)-1,2-cyclopropanedicarboxylic acid and 73.8 g of (−)-α-(1-naphthyl)ethylamine in 300 ml of tetrahydrofuran was diluted with 300 ml of ethyl ether and was allowed to stand at room temperature until crystallization is complete. The mixture was filtered and the crystals were collected and washed with cold tetrahydrofuran to give 4.95 g of a salt comprised of one molar equivalent of (+)-1-(p-tolyl)-1,2-cyclopropanedicarboxylic acid and one molar equivalent of (−)-α-(1-naphthyl)ethylamine. The salt was shaken with sodium hydroxide solution and ether. The aqueous phase was acidified with 12 N hydrochloric acid and the product was collected by filtration to give 26.0 g of (+)-1-(p-tolyl)-1,2-cyclopropanedicarboxylic acid as colorless crystals, [α]_{D}CH₃OH=+192°.

A 15.0 g portion of (+)-1-(p-tolyl)-1,2-cyclopropanedicarboxylic acid, 6.6 g of urea and 500 ml of xylene is refluxed and stirred for 5 hours. The reaction mixture was then filtered hot and the filtrate was evaporated under reduced pressure to give (+)-1-(p-tolyl)-1,2-cyclopropanedicarboximide as colorless crystals, m.p. 148°-155° C.

A 14 g portion of the above product was mixed with 420 ml of benzene and 112 ml of sodium bis(2-methoxyethoxy)aluminum hydride (70% benzene solution) was added over a 15 minute period with stirring. After refluxing for 1½ hours the mixture was cooled and 160 ml of 10 N sodium hydroxide was added. The organic layer was dried over sodium sulfate, filtered and evaporated to an oil. The oil was dissolved in ether and hydrogen chloride gas was bubbled in. The solid which forms was recrystallized from acetonitrile giving (+)-1-(p-tolyl)-3-azabicyclo[3.1.0]hexane hydrochloride as colorless crystals, m.p. 208°-210.5° C., [α]_{D}CH₃OH=+64.5°.

### EXAMPLE III

### Conversion of Racemic Bicifadine Hydrochloride to Polymorph Form B

Yet another alternative, exemplary bicifadine agent for use as an anti-incontinence drug was prepared according to the following protocol. Racemic bicifadine hydrochloride as a mixture of polymorphic forms A and B, was added to isopropyl alcohol in a sufficient quantity to form a slurry. The slurry was subjected to agitation, such as mixing, at a temperature less than 30° C. The product was isolated by filtration and dried at 50° C in vacuo until loss on drying of <1% was achieved. The material produced was bicifadine hydrochloride polymorphic form B.

### EXAMPLE IV

### Alternate Conversion of Racemic Bicifadine Hydrochloride to Polymorph Form B

An alternate conversion method to produce polymorph B of bicifadine for use as an anti-incontinence agent can be employed as follows. Twenty grams of racemic bicifadine hydrochloride as a mixture of polymorphic forms A and B were added to 50 ml of isopropyl alcohol to form a slurry. The slurry was stirred for 24 hours at a temperature of about 30° C. The product was isolated by filtration and dried in vacuo. The material produced was purified bicifadine hydrochloride polymorphic form B.

### EXAMPLE V

### Effects of Bicifadine Hydrochloride in Cats Using a Dilute Acetic Acid Model

Adult cats (male and female) were anesthetized with α-chloralose (50-75 mg/kg i.v.) A cannula was inserted into the trachea to maintain a clear airway. One catheter was inserted into the carotid artery to measure systemic blood pressure, and another was placed into the radial vein for injecting bicifadine hydrochloride. All doses of bicifadine hydrochloride were based on body weight.

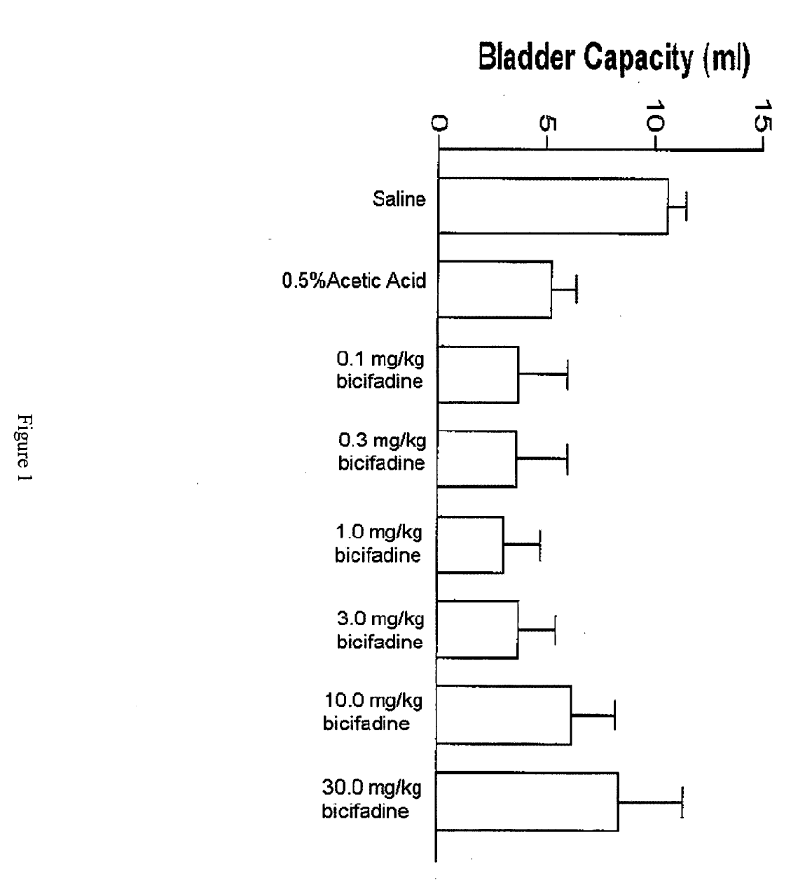

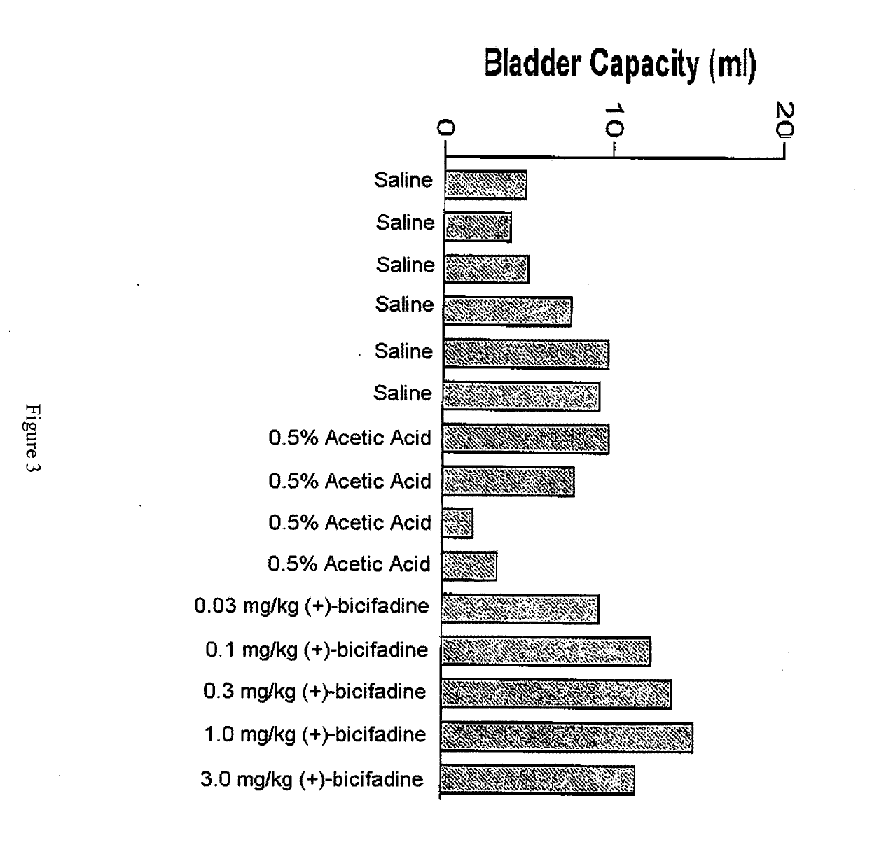

Electromyography electrodes were placed in the periurethral striated muscle. A catheter was inserted through the dome of the bladder and was used to infuse either saline for the control or dilute (0.5%) acetic acid into the test cats. The catheter was also used to record bladder pressure during each voiding cystometrogram. After bladder capacity was established using the saline infusion, cysometrograms were taken after an infusion of dilute acetic acid and changes in bladder capacity were recorded. (FIG. 1 and FIG. 3) Cystometrograms were repeated using either saline or acetic acid until bladder capacity measurements in three consecutive cystometograms had been recorded indicating that a stable base line had been achieved. Fluid release during micturition was measured by collecting the fluid in a cylinder attached to a force transducer.

On reaching micturition threshold, acetic acid infusion of the bladder was continued, resulting in rhythmic micturition contractions throughout the infusion period. During this time of rhythmic bladder activity, either vehicle as the control or bicifadine hydrochloride was administered. Five minutes after each administration of bicifadine hydrochloride, the bladder was emptied and another cystometrogram was performed. This procedure was repeated with increasing doses of bicifadine hydrochloride.

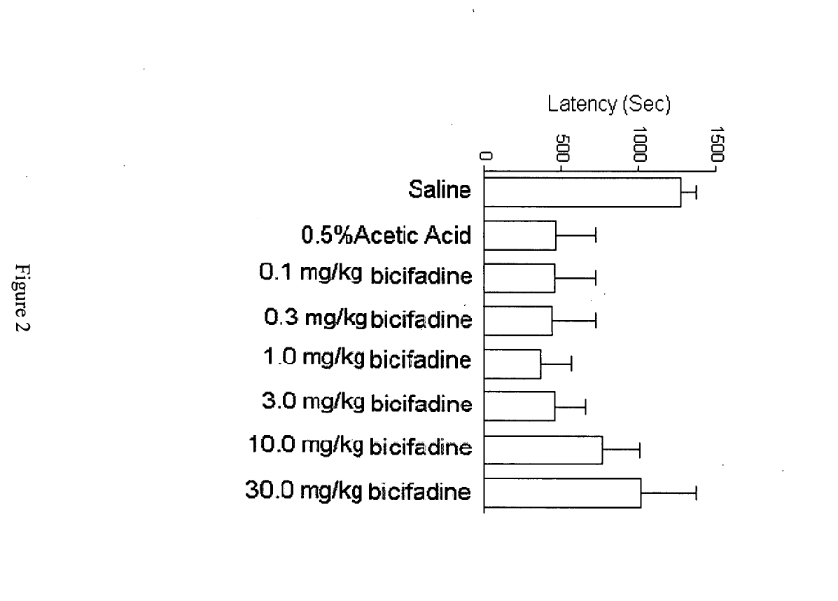

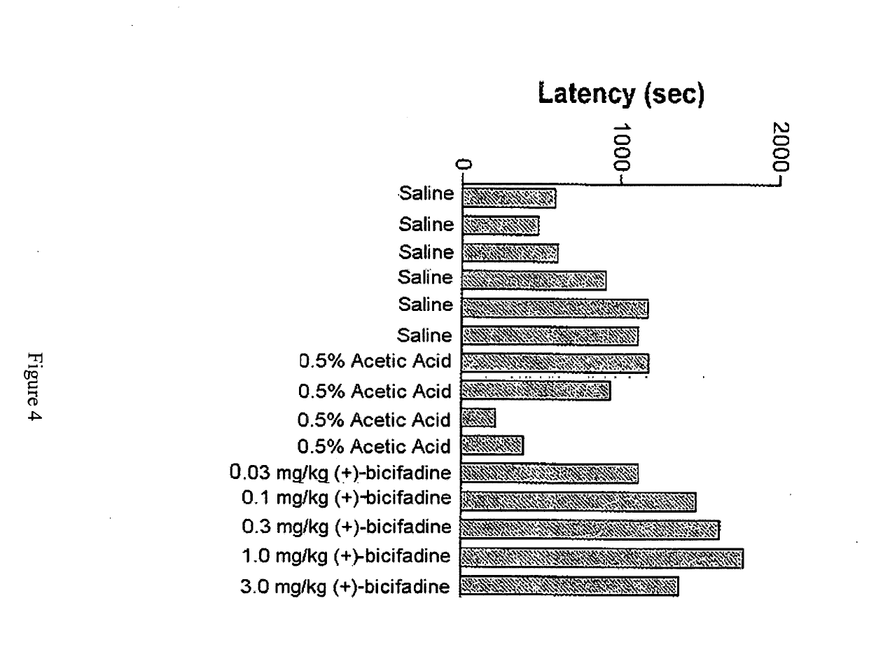

Under control conditions, rapid increases in intravessicular pressure were recorded after infusion of 5.3±0.6 ml saline into an initially empty bladder. Bladder capacity was reached after infusion of approximately 10.5 ml of saline. When acetic acid was infused, bladder capacity was reduced by more than half. Contraction amplitude and duration were also reduced, but contraction frequency increased. Normal time to contraction following instillation of saline into the bladder is approximately 1286 seconds. The administration of bicifadine hydrochloride (FIGS. 1 and 2) and (+)-bicifadine HCl (FIGS. 3 and 4) during acetic acid infusion yielded pronounced inhibition of bladder activity, increased the time to contraction, and increased bladder capacity to near control conditions, as depicted in FIGS. 1-4.

### EXAMPLE VI

### Effects of Bicifadine Hydrochloride in Rats Using a Dilute Acetic Acid Model

Additional animal models for evaluating efficacy of bicifadine as an anti-incontinence drug include a widely accepted rat model predictive of anti-incontinence drug activity in humans. Female rats (250-275 g BW, n=8) are anesthetized with urethane (1.2 g/kg) and a saline-filled catheter is inserted into the proximal duodenum for intraduodenal drug administration. A flared-tipped catheter is inserted into the bladder dome, via a midline lower abdominal incision, for bladder filling and pressure recording, and secured by ligation. Electromyography elctrodes are inserted into the external urethral sphincter percutaneously.

In the control, saline is continuously infused at a rate of about 0.055 ml/min via the bladder filling catheter for about 60 minutes to obtain a baseline of lower urinary tract activity. At the end of the control saline cystometry period, the infusion pump is stopped, the bladder is emptied and a single filling cystometrogram is performed using saline at the same flow rate as the continuous infusion, in order to measure bladder capacity. Bladder capacity (ml) is calculated as the flow rate of the bladder filling solution (m/min) multiplied by the elapsed time between commencement of bladder filling and occurrence of bladder contraction (min).

Following the control period, a 0.25% acetic acid solution in saline is infused into the bladder to induce bladder irritation. Following 30 minutes of acetic acid infusion, 3 vehicle injections (10% TWEEN®, 80 in saline, 1 ml/kg dose) are administered intraduodenally at 20 minute intervals to determine vehicle effects on the intercontraction interval and to achieve a stable level of irritation with the dilute acetic acid solution. Following injection of the third vehicle control, bladder capacity is again measured using acetic acid to fill the bladder. Increasing doses of bicifadine hydrochloride are then administered intraduodenally at 60 minute intervals in order to construct a cumulative dose-response relationship. Bladder capacity is measured as described above using acetic acid to fill the bladder, at 20 and 50 minutes following each subsequent drug treatment.

Bicifadine hydrochloride administration will yield an increase in bladder capacity in the dilute acetic acid model, as measured by filling cystometry in rats during continuous irritation.

### EXAMPLE VII

### Effects of Bicifadine Hydrochloride on Urethral Function in Guinea Pigs

Yet another animal model for evaluating efficacy of bicifadine as an anti-incontinence drug is a widely accepted guinea pig model, also predictive of anti-incontinence drug activity in humans. Adult female guinea pigs, weighing 620-707 g, are initially anesthetized with halothane and maintained with urethane. A cannula is inserted into the trachea, a jugular vein and a carotid artery for respiratory ventilation, injection of the test compound and monitoring of the blood pressure, respectively. A midline laporatomy is performed to expose the urinary bladder and a cystometry tube is inserted through a small incision in the dome of the bladder. The abdominal wound is closed tightly around the externalized cystometry tube, which is connected to an infusion pump and pressure transducer, for filling the bladder and recording intravesical pressure. Electromyographic electrodes are inserted into the striated muscles of the external urethral sphincter.

The bladder is filled at a rate of 150 μl/min⁻¹ with saline until initiation of a micturition reflex. The bladder is then drained and refilled three times to establish a bladder threshold capacity as well as electromyographic activity and intravesical pressure.

The bladder is then filed to 75% of the threshold volume with saline and weights are positioned on the ventral surface of the abdomen of the animal just rostral to the position of the bladder. Starting at 50 g, then 60 g and then at increasing increments of 20 g, weights are placed on the animal's abdomen until micturition/leakage of fluid is observed. Electromyographic activity and intravesical pressure are recorded while weights are applied to the abdomen. Once a base line is established, the bladder is emptied and refilled. Bicifadine hydrochloride or vehicle is injected intravenously immediately after the bladder is filled to the 75% of threshold volume, and 60-120 sec before applying the first abdominal weight (50 g). Weights are added until micturition/leakage of fluid is observed.

During normal bladder filling (150 μl/min⁻¹) the electromyographic activity increases gradually until micturition occurs, after which time activity returns to baseline level. Subsequent administration of bicifadine hydrochloride and repeated normal bladder filling will result in an increase in electromyographic activity above that recorded in the absence of drug or on administration of vehicle alone.

### EXAMPLE VIII

### Effects of Bicifadine Hydrochloride on Neurogenic Overactive bladder in Rats

In another animal model demonstrative of anti-incontinence activity, female rats (250-300 g) are anesthetized with isofluorane (4%) and a laminectomy is performed at the T9-10 spinal level. The spinal cord is transected and the intervening space filled with Gelfoam. The overlying muscle layers and skin are sequentially closed with suture, and the animals are treated with antibiotic (100 mg/kg ampicillin s.c.) Residual urine is expressed prior to returning the animals to their cages, and thereafter 3 times daily until terminal experimentation four weeks later. On the day of the experiment, the animals are anesthetized with isofluorane (4%) and a jugular catheter is inserted for access to the systemic circulation and tunneled subcutaneously to exit through the midscapular region. Via a midline abdominal incision, a catheter with a fire-flared tip is inserted into the dome of the bladder through a small cystotomy and secured by ligation for bladder filling and pressure recording. Electrodes are inserted percutaneously into the external urethral sphincter for electromyography readings. The abdominal wall and the overlying skin of the neck and abdomen are closed with suture and the animal is mounted in a Ballman-type restraint cage. A water bottle is positioned within easy reach of the animal's mouth for ad libitum access to water. Following a 30 minute recovery from anesthesia and acclimatization, normal saline is infused at a constant rate (0.100-0.150 ml/min) for control cystometric recording.

Following a 60-90 minute control period of normal saline infusion (0.100-0.150 ml/min) to collect baseline continuous open cystometric data, the pump is turned off, the bladder is emptied, the pump turned back on, and bladder capacity is estimated by a filling cystometrogram. At 3×20-30 minute intervals, vehicle is administered intravenously. Following the third administration of vehicle control, bladder capacity is again measured through a cystometrogram. Increasing doses of bicifadine hydrochloride are then administered intraduodenally at 60 minute intervals in order to construct a cumulative dose-response relationship. Bladder capacity is measured as described above at 20 minutes following each subsequent drug treatment. Administration of an effective amount of bicifadine hydrochloride to these model subjects under the foregoing test conditions will yield an increase in bladder capacity compared to that observed in the absence of drug or on administration of vehicle alone

### EXAMPLE IX

### Inhibition of Radioligand Binding and [³H]Biogenic Amine Reuptake by Bicifadine HCl

Further insight into the mechanism by which bicifadine HCl exerts its novel anti-incontinence activity was obtained by biochemical assays (Table 5, below). Bicifadine HCl was capable of inhibiting radioligand binding to the α₁ and α₂ adrenergic receptors, the 5-HT_{2A} serotonin receptor, and inhibiting the reuptake to both [³H]norepinephrine and [³H]serotonin by their respective transport proteins. Because both noradrenergic and serotonergic pathways have been implicated in the control of bladder function, these biochemical actions, either individually or in concert, could contribute to the pharmacological actions of bicifadine.

For the α₁ adrenergic receptor, the ability of bicifadine to inhibit the binding of [³H]prazosin to receptors in a rat cerebral cortex preparation was investigated using a modification of the technique of Greengrass and Bremner (1979). The rat cortex preparation was incubated with a 0.25 nM concentration of [³H]prazosin for 60 min at 22° C. with either 0.1, 0.3, 1, 3, or 10 μM concentrations bicifadine HCl. Nonspecific binding was determined using 0.5 μM unlabelled prazosin. At the end of 60 min, the assay was terminated by vacuum filtration and the amount of radioactivity deposited on the filter measured by scintillation counting. Bicifadine HCl bound to the α₁ adrenergic receptor with an affinity (Kᵢ) of 1 μM (Table 5), while the reference agent, prazosin bound with an affinity (Kᵢ) of 0.15 nM (not shown).

Similarly, the ability of bicifadine to inhibit the binding of [³H]RX 821002 to α₂ receptors in a rat cerebral cortex preparation was demonstrated using a modification of the technique of Uhlen and Wikberg (1991). This assay was conducted by incubating the rat cortex preparation with a 0.5 nM concentration of [³H]RX 821002 for 30 min at 22° C. in the presence of either 0.1, 0.3, 1, 3, or 10 μM concentrations bicifadine HCl. Unlabelled (−)epinephrine (100 μM) was used to determine nonspecific binding. The assay was terminated by vacuum filtration, and the amount of radiolabeled receptor retained by the filter measured by scintillation counting. Bicifadine HCl bound to the α₂ adrenergic receptor with an affinity (Kᵢ) of 610 nM (Table 5), while the reference agent, yohimbine, bound with an affinity (Kᵢ) of 28 nM. Finally, bicifadine HCl was found to interact with the 5-HT_{2A} receptor in a receptor binding screen. Recombinant human 5-HT_{2A} receptors expressed in CHO cells were incubated with bicifadine HCl (0.1, 1 or 10 μM) and [³H]LSD (1.2 nM) at 37° C. for 30 min (Bonhaus 1995). Serotonin (10 μM) was used to determine nonspecific binding. Following termination of the assay, it was found that bicifadine HCl maximally inhibited radioligand binding to the 5-HT_{2A} receptor by 82% at a concentration of 10 μM (Table 5).

The effects bicifadine on [³H]norepinephrine and [³H]serotonin uptake were measured in HEK 293 cells expressing the recombinant human forms of the norepinephrine and serotonin transporter proteins, respectively using a modification of the techniques described by Eschleman, et al., (1999). Cells were grown on 150-mm-diameter tissue culture dishes until confluent. Medium was removed from the plates and the cells were washed twice with Ca²⁺, Mg²⁺-free PBS. Fresh Ca²⁺, Mg²⁺-free PBS (2.5 ml) was then added to each plate and the plates were placed in a 25° C. water bath for 5 min. The cells were then gently scraped from the plates, and cell clusters were separated by trituration with a pipette for 5 to 10 aspirations and ejections. Aliquots (50 μl) of the suspended cells were then added to assay tubes (in triplicate) containing various concentrations of bicifadine and Krebs-HEPES assay buffer in a final assay volume of 0.5 ml. Following a 10-min pre-incubation in a 25° C. water bath, [³H]norepinephrine or serotonin (20 nM final concentration) was added, and the assay was incubated for 10 min. The reaction was terminated by filtration through Wallac filtermat A filters presoaked in 0.05% polyethylenimine, using a Tomtec cell harvester. Scintillation fluid was then added to each filtered spot, and radioactivity remaining on the filters was determined by scintillation counting. Specific uptake was defined as the difference in uptake observed in the absence and presence of 5 μM mazindol (for measurement of [³H]norepinephrine reuptake) or 5 μM imipramine (for measurement of [³H]serotonin uptake, respectively). As can be seen in Table 5, bicifadine HCl inhibited the reuptake of both biogenic amines.

TABLE 5
Interaction of bicifadine with monoaminergic
neurotransmitter receptors and transporters.

| | Inhibition of Radioligand Binding or |
|---|---|
| Receptor System | inhibition of [³H]amine uptake |
| α₁ Adrenergic | 1 μM (Kᵢ) |
| α₂ Adrenergic | 600 nM (Kᵢ) |
| 5-HT_{2A} | 82% inhibition at 10 μM |
| [³H]Norepinephrine uptake | 54.7 nM (IC₅₀) |
| [³H]Serotonin uptake | 117 nM (IC₅₀) |

Results were obtained from competition radioligand binding assays, involving the displacement of radioligands selective for the indicated receptors from their binding sites by bicifadine. Kᵢ=inhibitory constant defined as IC₅₀/L+K_{D}; IC₅₀=concentration of bicifadine HCl that inhibits 50% of the maximal response; L=concentration of radioligand added; K_{D}, dissociation constant of the radioligand at equilibrium.

The interaction of bicifadine HCl with this combination of receptors and transport proteins may contribute to its anti-incontinence profile. Animal studies have suggested the involvement of 5-HT containing neurons in sending projections to the dorsal horn as well as to the autonomic and sphincter motor nuclei in the lumbosacral spinal cord. It is further predicted that activity in the serotonergic pathway enhances urine storage by facilitating the vesical sympathetic reflex pathway and inhibiting the parasympathetic-voiding pathway. The 5-HT2 and 5-HT3 receptors mediate the excitatory effects on sympathetic and somatic reflexes resulting in increased outlet resistance. The ability of bicifadine to inhibit serotonin reuptake (and thereby produce a higher synaptic concentration of this transmitter) would activate these serotonin receptors.

From these and additional observations, the side-effect profile attending the use of bicifadine HCl as an anti-incontinence agent will be significantly narrowed and reduced compared to other anti-incontinence agents. For example, the rate of occurrence and/or severity of most common side effects of anti-incontinence drugs following administration of an anti-incontinence effective dose of bicifadine HCl will often be below 95% or less, 75% or less, 50% or less, 25-30% or less, and as low as 5-10% or less, compared to the rate of occurrence and/or severity of these side effects following administration of other conventional anti-incontinence agents as described above. In addition, the ability to inhibit norepinephrine uptake renders the bicifadine formulations and methods of the invention safer in terms of a comparably reduced or eliminated occurrence of vasodilation, hypotension and other related adverse symptoms elicited by selective α₁ andrenergic antagonists. This improved characteristic of the inventive compositions and methods herein is evidenced by the finding that bicifadine HCl did not cause significant alterations in blood pressure in human subjects. In selected embodiments of the invention, the rate of occurrence and/or severity of vasodilation and/or hypotension following administration of an anti-incontinent effective dose of bicifadine will be below, often 95% or less, 75% or less, 50% or less, 25-30% or less, and as low as 5-10% or less, compared to the rate of occurrence and/or severity of these side effects following administration of an anti-incontinent effective dose of a selective α₁ andrenergic antagonist.

Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited within the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

### REFERENCES

Eschleman A, Carmolli M, Cumbay M, Martens C, Neve K and Janowsky A. Characteristics of Drug Interactions with Recombinant Biogenic Amine Transporters Expressed in the Same Cell Type. J. Pharmacol Exp. Ther. 289:877-885, (1999).
Greengrass P, Bremner R. Binding characteristics of 3H-prazosin to rat brain alpha-adrenergic receptors. Eur J Pharmacol. 55(3):323-6 (1979).
Sharma, A. et al., Pharmacokinetics and Safety of Duloxetine, a Dual-Serotonin andn Norepinephrine Reuptake Inhibitor. J. Clin. Pharmacol., 40:161-167 (2000).
Thor K B, Katofiasc M A. Effects of duloxetine, a combined serotonin and norepinephrine reuptake inhibitor, on central neural control of lower urinary tract function in the chloralose-anesthetized female cat. J Pharmacol Exp Ther. 274(2):1014-24, (1995).
Uhlen S, Wikberg J E. Delineation of rat kidney alpha 2A- and alpha 2B-adrenoceptors with [3H]RX821002 radioligand binding: computer modelling reveals that guanfacine is an alpha 2A-selective compound. Eur J Pharmacol. 202(2):235-43, (1991).

1. A method for preventing or treating lower urinary tract disorders in a mammalian subject comprising administering an anti-incontinence effective amount of bicifadine to said subject.

2. The method of claim 1, wherein the bicifadine comprises bicifadine hydrochloride.

3. The method of claim 2, wherein the bicifadine comprises bicifadine polymorph A, bicifadine polymorph B, or a mixture of A and B polymorphs of bicifadine hydrochloride.

4. The method of claim 1, further comprising administering a second anti-incontinence agent to said subject.

5. The method of claim 4, wherein the second anti-incontinence agent is administered to said subject in a combined formulation with said bicifadine.

6. The method of claim 4, wherein the second anti-incontinence agent is administered to said subject in a coordinate administration protocol, simultaneously with, prior to, or after, administration of said bicifadine to the subject.

7. The method of claim 4, wherein the second anti-incontinence agent is selected from α2δ subunit calcium channel modulators, 4-phenyl substituted tetrahydroisoquinolines, 5-HT3 receptor antagonists, 5-α reductase inhibitors, antibiotics, anticholinergic drugs, anticonvulsants, antidepressants, antihistamines, antimuscarinics, antispasmodics, bradykinin receptor agonists, buprenorphine, calcium antagonists, cox-2 inhibitors, dibenzazepines, hormones, hydantoins, muscle relaxants, noradrenaline reuptake inhibitors, nitric oxide donors, neurokinin receptor agonists, NSAIDS, parasympatholytics, potassium channel openers, prostaglandin synthesis inhibitors sodium channel modulators, vasopressin analogues, α-adrenoreceptor antagonists, and β-adrenoreceptor agonists.

8-35. (canceled)

36. The method of claim 1, further comprising an additional therapeutic treatment selected from the group consisting of diet modification, bladder training, pelvic floor training, muscle awareness, muscle training, biofeedback, behavioral modification, bladder reflex triggering, electrical stimulation and surgery.

37. The method of claim 1, wherein said lower urinary tract disorder is neurogenic overactive bladder, non-neurogenic overactive bladder, interstitial cystitis, prostatitis, prostadynia, or benign prostatic hyperplasia.

38. The method of claim 1, wherein said method effectively prevents or alleviates urinary incontinence.

39. The method of claim 38, wherein said urinary incontinence includes urge incontinence, stress incontinence, overflow incontinence, functional incontinence, neurogenic incontinence, post-prostatectomy incontinence, urinary urgency, nocturia, or enuresis.

40. (canceled)

41. The method of claim 1, wherein said anti-incontinence effective amount comprises between about 70 to about 1,800 mg of bicifadine per day.

42. (Cancelled).

43. The method of claim 1, wherein said anti-incontinence effective amount comprises between about 25 mg to about 500 mg of bicifadine.

44. (canceled)

45. The method of claim 1, wherein said anti-incontinence effective amount of bicifadine is administered one, two, three, or four times per day.

46-53. (canceled)

54. A method of controlling the lower urinary tract in a mammalian subject to reduce or prevent uncontrolled loss of urine comprising administering to said subject an anti-incontinence effective amount of bicifadine.

55. The method of claim 54, wherein the method is effective to prevent or alleviate one or more conditions of urge incontinence, stress incontinence, overflow incontinence, functional incontinence, neurogenic incontinence, post-prostatectomy incontinence, urinary urgency, nocturia, and/or enuresis in said subject.

56. The method of claim 54, wherein the uncontrolled loss of urine is associated with neurogenic overactive bladder, non-neurogenic overactive bladder, interstitial cystitis, prostatitis, prostadynia, or benign prostatic hyperplasia in said subject.

57. The method of claim 54, wherein the uncontrolled loss of urine is associated with Parkinson's disease, multiple sclerosis, muscle disease, diabetes, spinal cord injury, nerve disorders of the pelvic floor, a congenitally short urethra, damage to the sphincter from surgery, damage from childbirth, weight gain, urinary tract infections, bladder stones, hormonal imbalances, destruction of the sensory nerve fibers, inflammatory conditions, certain medications, or weakness of certain muscles in said subject.

58-62. (canceled)

63. The method of claim 54, wherein the effective amount is between about 70 to about 1,800 mg of bicifadine per day.

64. (canceled)

65. The method of claim 54, wherein said anti-incontinence effective amount is between about 25 mg to about 500 mg of bicifadine.

66-74. (canceled)

75. A method of treating one or more symptoms of urinary incontinence comprising administering to a mammalian subject an effective amount of bicifadine.

76. The method of claim 75, wherein said one or more symptoms of urinary incontinence is/are selected from leakage, urgency, frequency, overflow, nocturnal urgency, and bed wetting.

77. (canceled)

78. A composition for preventing or alleviating incontinence in a mammalian subject comprising an anti-incontinence effective amount of bicifadine or a pharmaceutically-acceptable salt, isomer, enantiomer, solvate, hydrate, polymorph or prodrug thereof.

79. A composition for treating or preventing incontinence in a mammalian subject comprising an anti-incontinence effective amount of bicifadine, and an adjunctive agent selected from a second anti-incontinence agent and other adjunctive therapeutic agents useful in the treatment of a lower urinary tract disorder.

80. The composition of claim 79, wherein the second anti-incontinence agent is selected from α2δ subunit calcium channel modulators, 4-phenyl substituted tetrahydroisoquinolines, 5-HT3 receptor antagonists, 5-α reductase inhibitors, antibiotics, anticholinergic drugs, anticonvulsants, antidepressants, antihistamines, antimuscarinics, antispasmodics, bradykinin receptor agonists, buprenorphine, calcium antagonists, cox-2 inhibitors, dibenzazepines, hormones, hydantoins, muscle relaxants, noradrenaline reuptake inhibitors, nitric oxide donors, neurokinin receptor agonists, NSAIDS, parasympatholytics, potassium channel openers, prostaglandin synthesis inhibitors sodium channel modulators, vasopressin analogues, α-adrenoreceptor antagonists, and β-adrenoreceptor agonists.

81. The composition of claim 78, wherein said anti-incontinence effective amount comprises between about 20 mg to about 1,800 mg of bicifadine.

82. The composition of claim 78, wherein said anti-incontinence effective amount comprises between about 25 mg to about 500 mg of bicifadine.

83-91. (canceled)

92. A composition for treating one or more symptoms of overactive bladder, interstitial cystitis, prostatitis, prostadynia, or benign prostatic hyperplasia in a mammalian subject comprising an anti-incontinence effective amount of bicifadine.

93-94. (canceled)
